(19) United States
(12) Patent Application Publication
(10) Pub. No.: US 2022/0148712 A1
(43) Pub. Date: May 12, 2022

(54) RADIATION IMAGING SYSTEM AND RADIATION IMAGING METHOD

(71) Applicant: NanoRay Biotech Co., Ltd., Taipei City (TW)

(72) Inventor: Wen-Yuan Cheng, Taipei City (TW)

(73) Assignee: NanoRay Biotech Co., Ltd., Taipei City (TW)

(21) Appl. No.: 17/501,991

(22) Filed: Oct. 14, 2021

Related U.S. Application Data
(60) Provisional application No. 63/110,371, filed on Nov. 6, 2020.

(30) Foreign Application Priority Data
Aug. 27, 2021 (TW) 110131887

(51) Int. Cl. G16H 30/20; A61B 6/00; G16H 50/20

(52) U.S. Cl. CPC G16H 30/20; A61B 6/548; A61B 6/4283; A61B 6/505; A61B 6/463; A61B 6/563; G16H 50/20; A61B 6/4035

(57) ABSTRACT

A radiation imaging system and a radiation imaging method are provided. The radiation imaging system includes a remote-control module and an imaging device. The imaging device has a radiation isolation cavity. The radiation isolation cavity includes a radiation irradiation area adapted for placing an object under test. The imaging device includes a controller, a radiation source, and a flat panel detector. The radiation source is disposed on a top of the radiation isolation cavity and faces the radiation irradiation area. The flat panel detector is disposed below the radiation exposure area. During a preparation for exposure, the controller turns on the radiation source. When the controller receives an activation signal output by the remote-control module, the controller operates the flat panel detector to obtain a radiation image corresponding to the object under test.

### CROSS-REFERENCE TO RELATED APPLICATION

This application claims the priority benefit of U.S. provisional application Ser. No. 63/110,371, filed on Nov. 6, 2020, and Taiwan application serial no. 110131887, filed on Aug. 27, 2021. The entirety of each of the above-mentioned patent applications is hereby incorporated by reference herein and made a part of this specification.

### BACKGROUND

### Technical Field

The disclosure relates to an imaging technology, and in particular to a radiation imaging system and a radiation imaging method.

### Description of Related Art

X-ray images are a common diagnostic tool in modern radiology. However, due to the danger of X-ray radiation, an X-ray image must be taken in a workplace that meets specific specifications. As a result, the shooting of X-ray images is limited to the site and further requires a certain cost to construct and maintain the safety of the workplace. In addition, a traditional X-ray imaging system has disadvantages including the apparatus being bulky and the system requiring a lot of manpower to set up.

### SUMMARY

The disclosure provides a radiation imaging system and a radiation imaging method, which easily and quickly obtain a radiation image of an object under test.

A radiation imaging system of the disclosure includes a remote-control module and an imaging device. The imaging device has a radiation isolation cavity. The radiation isolation cavity includes a radiation irradiation area adapted for placing an object under test. The imaging device includes a controller, a radiation source, and a flat panel detector. The controller is coupled to the remote-control module. The radiation source is coupled to the controller and is disposed on a top of the radiation isolation cavity. The radiation source faces the radiation irradiation area. The flat panel detector is coupled to the controller and is disposed below the radiation irradiation area. During a preparation for exposure, the controller turns on the radiation source. When the controller receives an activation signal output by the remote-control module, the controller operates the flat panel detector to obtain a radiation image corresponding to the object under test.

A radiation imaging method of the disclosure is adapted for a radiation imaging system. The radiation imaging system includes a remote-control module and an imaging device. The imaging device has a radiation isolation cavity. The radiation isolation cavity includes a radiation irradiation area adapted for placing an object under test. The radiation imaging method includes the following. During a preparation for exposure, a controller turns on a radiation source disposed on a top of the radiation isolation cavity. During the preparation for exposure, the remote-control module outputs an activation signal. When the controller receives the activation signal, the controller operates a flat panel detector disposed below the radiation irradiation area to obtain a radiation image corresponding to the object under test.

Based on the above, in the radiation imaging system and the radiation imaging method of the disclosure, the radiation source irradiates the object under test placed on the radiation irradiation area in the radiation isolation cavity, and the flat panel detector obtains the radiation image corresponding to the object under test. The radiation imaging system and the radiation imaging method of the disclosure may realize convenient and effective imaging functions of a radiation image.

To provide a further understanding of the above features and advantages of the disclosure, embodiments accompanied with drawings are described below in details.

### BRIEF DESCRIPTION OF THE DRAWINGS

FIG. 1 is a schematic circuit diagram of a radiation imaging system according to an embodiment of the disclosure.

FIG. 2 is a flow chart of a radiation imaging method according to an embodiment of the disclosure.

FIG. 3 is a schematic circuit diagram of a radiation imaging system according to another embodiment of the disclosure.

FIG. 4 is a structural side view of an imaging device according to an embodiment of the disclosure.

FIG. 5 is a schematic view of a radiation imaging system according to another embodiment of the disclosure.

FIG. 6 is a flow chart of a radiation imaging method according to another embodiment of the disclosure.

FIG. 7A is a schematic view of a login interface according to an embodiment of the disclosure.

FIG. 7B is a schematic view of a diagnostic object information interface according to an embodiment of the disclosure.

FIG. 7C is a schematic view of an operation interface according to an embodiment of the disclosure.

FIG. 7D is a schematic view of another operation interface according to an embodiment of the disclosure.

FIG. 7E is a schematic view of an operation interface according to yet another embodiment of the disclosure.

FIG. 7F is a schematic view of an image processing interface according to an embodiment of the disclosure.

### DESCRIPTION OF THE EMBODIMENTS

To provide a further understanding of the content of the disclosure, embodiments as examples of how this disclosure may be implemented are described below. In addition, wherever possible, elements/components/steps with the same reference numeral in the drawings and embodiments represent the same or similar components.

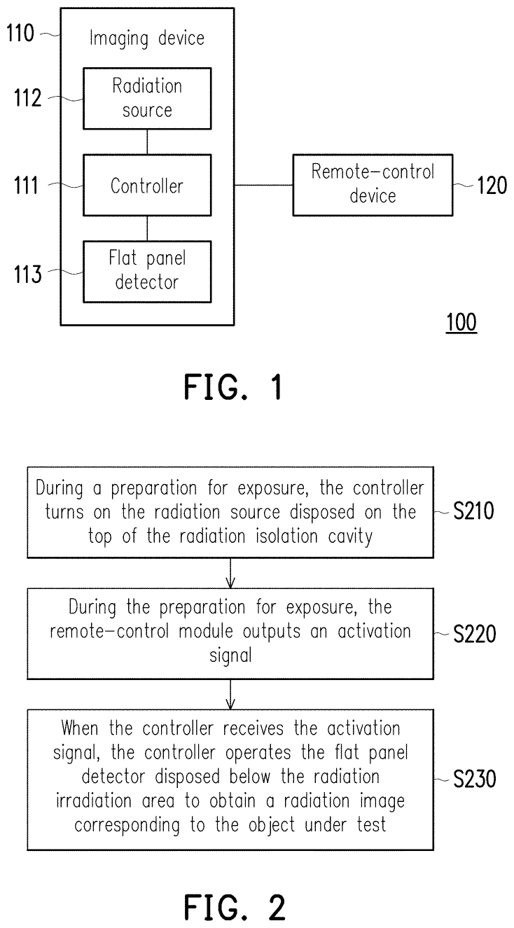

FIG. 1 is a schematic circuit diagram of a radiation imaging system according to an embodiment of the disclosure. Referring to FIG. 1, a radiation imaging system 100 includes an imaging device 110 and a remote-control device 120. The imaging device 110 is coupled to the remote-control device 120. The imaging device 110 may be electrically connected to the remote-control device 120 in a wired or wireless manner. In this embodiment, the imaging device 110 may have a radiation isolation cavity. The radiation isolation cavity includes a radiation irradiation area adapted for placing an object under test. In this embodiment, the imaging device 110 includes a controller 111, a radiation source 112, and a flat panel detector (FPD) 113. The controller 111 is coupled to the remote-control module 120, the radiation source 112 and the flat panel detector 113. The radiation source 112 may be disposed on a top of the radiation isolation cavity. The radiation source 112 may face the radiation irradiation area. The flat panel detector 113 is disposed below the radiation irradiation area. In this embodiment, the radiation source 112 may be an X-ray light source, and the flat panel detector 113 may be an X-ray flat panel sensor. The flat panel detector 113 may generate an X-ray radiation image with a large image range.

In this embodiment, the controller 111 may include a central processing unit (CPU), a microprocessor, a digital signal processor (DSP), a programmable controller, a programmable logic device programmable logic device (PLD) or other similar devices or combinations of these devices, and the disclosure is not limited thereto. The radiation imaging system 100 may further include a storage device, such as a memory. The storage device may be used to store a related control software, operate commands, image data, etc., but the disclosure is not limited thereto. The controller 111 is coupled to the storage device, and may access the related data stored in the storage device, so as to implement the related operations described in each embodiment of the disclosure.

FIG. 2 is a flow chart of a radiation imaging method according to an embodiment of the disclosure. Referring to FIGS. 1 and 2, the radiation imaging system 100 of this embodiment may execute the following steps S210 to S230. In this embodiment, the subject may place the object under test in the radiation irradiation area in the radiation isolation cavity, and the object under test may be, for example, the subject's hand (which may include the palm and/or wrist). Next, in step S210, during a preparation for exposure, the controller 111 turns on the radiation source 112 disposed on the top of the radiation isolation cavity. In step S220, during the preparation for exposure, the remote-control module 120 outputs an activation signal. In step S230, when the controller 111 receives the activation signal, the controller 111 may operate the flat panel detector 113 disposed below the radiation irradiation area to obtain a radiation image corresponding to the object under test. Therefore, the radiation imaging system and the radiation imaging method of the disclosure may realize convenient and effective radiation image imaging functions.

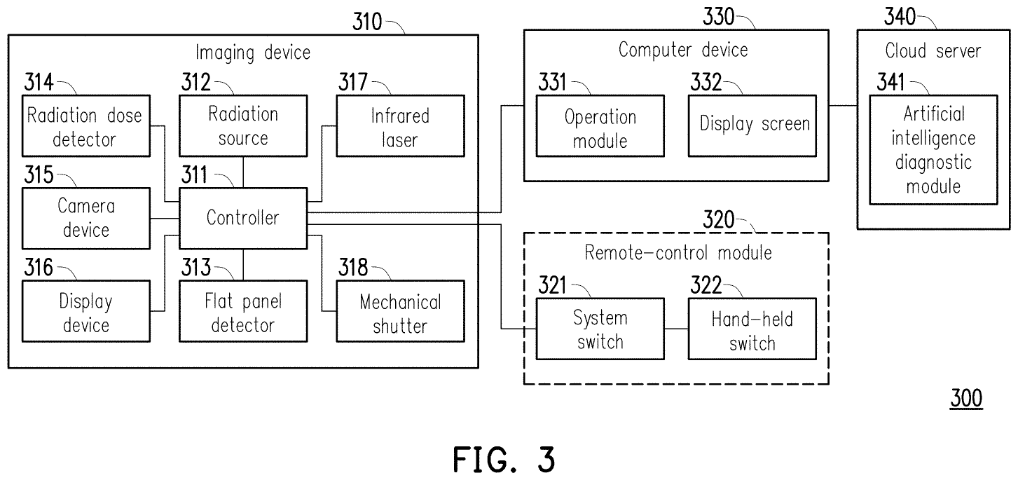

FIG. 3 is a schematic circuit diagram of a radiation imaging system according to another embodiment of the disclosure. Referring to FIG. 3, a radiation imaging system 300 includes an imaging device 310, a remote-control module 320, a computer device 330, and a cloud server 340. In this embodiment, the imaging device 310 includes a controller 311, a radiation source 312, a flat panel detector 313, a radiation dose detector 314, a camera device 315, a display device 316, an infrared laser 317, and a mechanical shutter 318. The controller 311 is coupled to the radiation source 312, the flat panel detector 313, the radiation dose detector 314, the camera device 315, the display device 316, the infrared laser 317, and the mechanical shutter 318. In this embodiment, the imaging device 310 is coupled to the remote-control module 320 and the computer device 330. The computer device 330 is coupled to the cloud server 340. The imaging device 310 may be connected to the remote-control module 320 and the computer device 330 in a wired or wireless manner. The imaging device 310 may transmit a radiation image to the computer device 330. The computer device 330 may be connected to the cloud server 340 in a wired or wireless manner to transmit detection data to the cloud server 340.

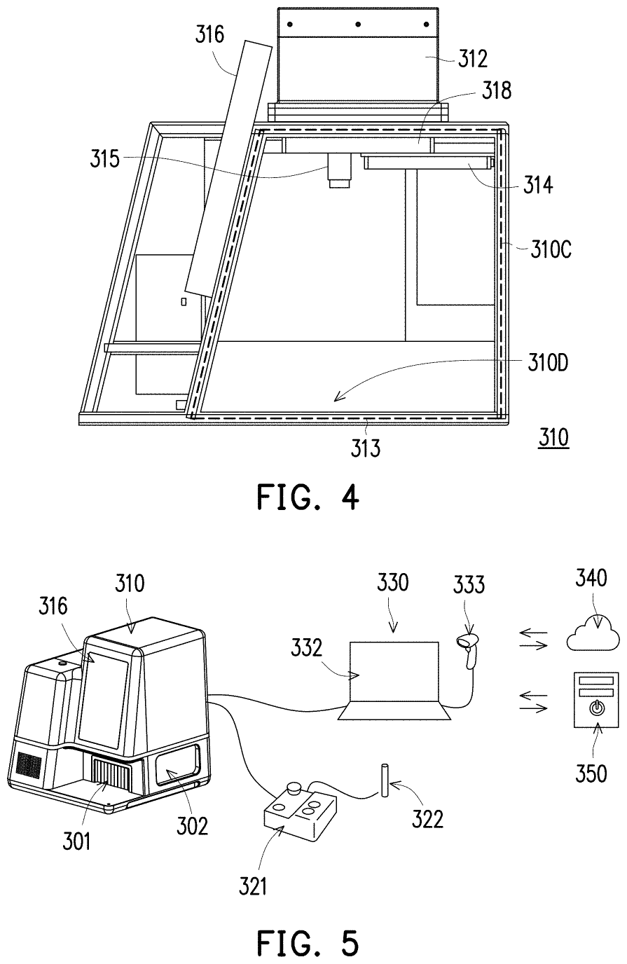

Referring to FIGS. 3 and 4, FIG. 4 is a structural side view of an imaging device according to an embodiment of the disclosure. In this embodiment, the imaging device 310 may be implemented as the structural form shown in FIG. 4, but the disclosure is not limited thereto. In this embodiment, the imaging device 310 may have a radiation isolation cavity 310C, and the radiation isolation cavity 310C includes a radiation irradiation area 310D adapted for placing an object under test. As shown in FIG. 4, the radiation source 312, the radiation dose detector 314, the camera device 315, and the mechanical shutter 318 of this embodiment may be disposed on the top of the radiation isolation cavity 310C. The flat panel detector 313 may be disposed below the radiation irradiation area 310D. In addition, the display device 316 may be disposed on a case of the imaging device 310 to provide a display function. The radiation source 312, the radiation dose detector 314, and the camera device 315 may face the radiation irradiation area 310D. In this embodiment, the radiation isolation cavity 310C may be formed by a plurality of lead components (such as lead plates, lead glass, etc.) to provide radiation isolation effects. In addition, the controller 311 of the present embodiment may be disposed at any circuit placement position in the imaging device 310, and the infrared laser 317 may be disposed at any position on the top of the radiation isolation cavity 310C, and the disclosure is not limited thereto.

In this embodiment, the radiation source 312 may be a continuous X-ray light source. The controller 311 may adjust the time length and the light output angle range of an X-ray beam emitted by the radiation source 312 to the radiation irradiation area 310D through operating the mechanical shutter 318. In this embodiment, the radiation dose detector 314 may be used to operate synchronously with the flat panel detector 313. When the flat panel detector 313 obtains the radiation image, the radiation dose detector 314 may obtain the radiation dose information corresponding to the radiation image of a single shot at the same time.

It is worth noting that the imaging device 310 may be a desktop hand diagnostic radiography imaging system, and may be applied, for example, to perform smart children's bone age detection, fracture detection, bone density detection, and gout detection. Therefore, the imaging device 310 of the embodiment may be designed as a desktop detection apparatus with a smaller volume. In this regard, the radiation source 312 and the flat panel detector 313 of this embodiment may have a shorter source image distance (SID) in between; for example, the source image distance may be about 45 centimeters (cm) (a traditional hand X-ray imaging distance is about 1 meter (m)). Therefore, the X-ray beam emitted by the radiation source 312 of the present embodiment during one detection process may have a lower X-ray dose, for example, the X-ray dose may be about 25 uGy (the dose of a traditional hand X-ray is about 50 uGy). In other words, the imaging device 310 of this embodiment may obtain a clear X-ray radiation image of a hand or other objects under test through a lower dose.

In this embodiment, the camera device 315 may be a camera of a charge-coupled device (CCD). The camera device 315 faces the radiation irradiation area 310D, and is used to obtain a video image corresponding to the object under test. In this embodiment, the display device 316 may display the video image. Moreover, in this embodiment, the infrared laser 317 may emit a registration pattern (such as a cross pattern) toward the radiation irradiation area 310D, so that the video image includes the registration pattern. In other words, the imaging device 310 of this embodiment allows the subject to observe the position and posture of the hand placed on the radiation irradiation area 310D in real time through the display device 316, and adjust the position and posture of the hand according to the registration pattern, so that the flat panel detector 313 may capture the correct or appropriate radiation image. In addition, the case of the imaging device 310 of this embodiment may have an emergency stop button disposed thereon.

FIG. 5 is a schematic view of a radiation imaging system according to another embodiment of the disclosure. Referring to FIGS. 3 to 5, in this embodiment, the remote-control module 320 includes a system switch 321 and a hand-held switch 322. As shown in FIG. 5, the remote-control module 320 is disposed outside the imaging device 310. The system switch 321 may be connected to the imaging device 310 in a wired or wireless manner, and the hand-held switch 322 may be coupled to the system switch 321 in a wired manner. In this embodiment, the system switch 321 is used to provide a switching signal to turn on the imaging device 310 through the switching signal. The hand-held switch 322 is used to provide an activation signal. In other words, in the shooting operation of the embodiment, the subject or the operator may turn on and activate the imaging device 310 to execute the imaging operation through operating the remote-control module 320. For example, the subject may be, for example, a child, a patient, or an ordinary person, and the operator may be, for example, a doctor or a nurse. Next, the subject may put his hand into the radiation isolation cavity 310C of the imaging device 310 from a cavity opening 301, and the subject and the operator may view the position and posture of the hand through the display device 316 or a lead glass 302 on a side of the radiation isolation cavity 310C near the radiation irradiation area 310D. In addition, when the subject places the hand correctly in the radiation isolation cavity 310C of the imaging device 310, the operator may remotely operate the imaging device 310 to perform an exposure operation to obtain a radiation image of the subject's hand.

In this embodiment, the computer device 330 may be a related computer apparatus such as a notebook computer or a tablet computer, and the disclosure is not limited thereto. The computer device 330 may include an operation module 331 and a display screen 332. The computer device 330 is used to execute the operation module 331 and displays an operation interface on the display screen 332. In this embodiment, the computer device 330 may further include a processor and a memory, and the memory may pre-store a related program and algorithm of the operation module 331 for the processor to execute. The operation module 331 may be a specific user operation interface and an image processing software. In this embodiment, the imaging device 310 may transmit the video image and the radiation image to the computer device 330. In this regard, the computer device 330 may first display the video image in the operation interface displayed on the display screen 332 during the preparation for exposure. Next, after the flat panel detector 313 obtains the radiation image, the computer device 330 may switch to display the radiation image in the operation interface displayed on the display screen 332. In addition, the operation interface may further include display radiation dose information, whole machine state information, and radiation setting parameter information.

In addition, in an embodiment, the computer device 330 may further be coupled to a bar code scanner 333 to, for example, obtain the corresponding diagnostic object information by scanning the bar code on a relevant medical record or body data of the subject. Therefore, the computer device 330 of this embodiment may perform image processing on the radiation image, and perform a profiling operation based on the diagnostic object information and the radiation image to generate a detection file.

In this embodiment, the cloud server 340 may include an artificial intelligence diagnostic module 341. The computer device 330 may, for example, transmit the detection file to the cloud server 340 via the Internet, so that the artificial intelligence diagnostic module 341 of the cloud server 340 may analyze the radiation image and the diagnostic object information in the detection file to automatically generate corresponding diagnostic data. However, as shown in FIG. 5, the computer device 330 may further be connected to a picture archiving and communication system (PACS) 350 to transmit the detection file with the radiation image to the picture archiving and communication system 350 for relevant medical personnel to obtain the detection file through the picture archiving and communication system 350 and perform diagnosis.

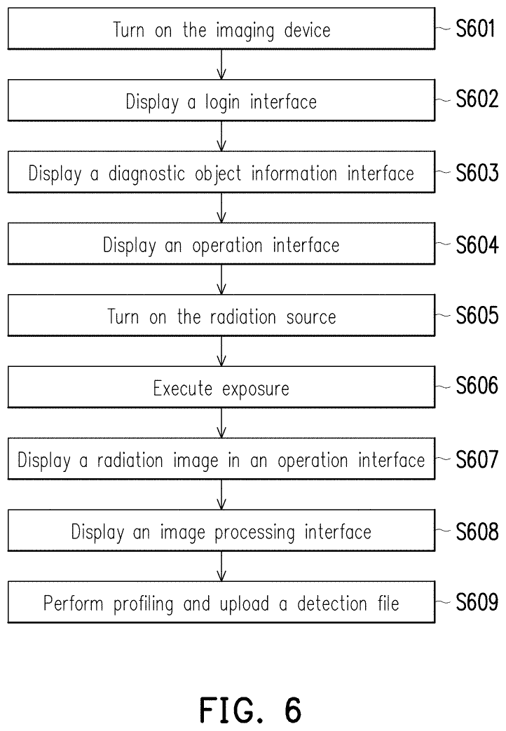

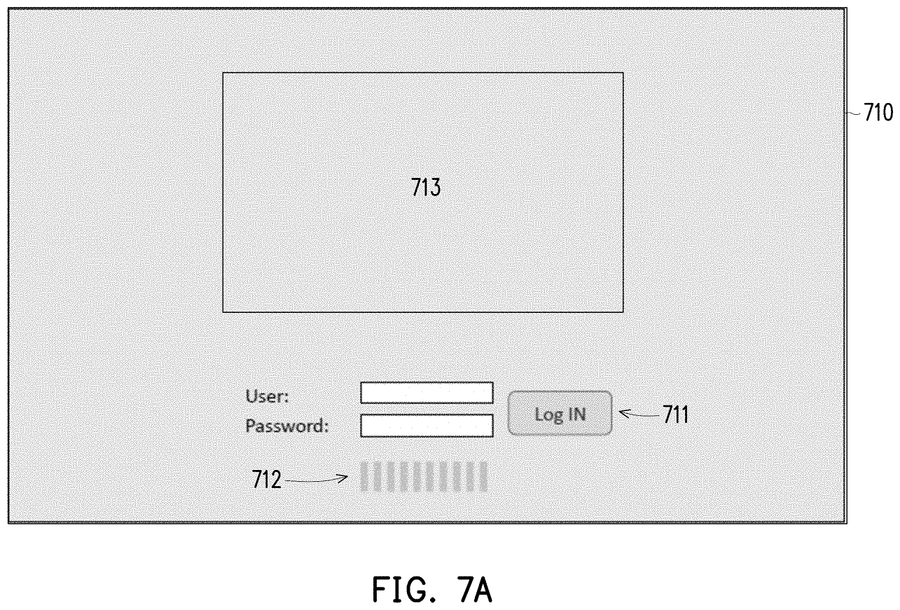

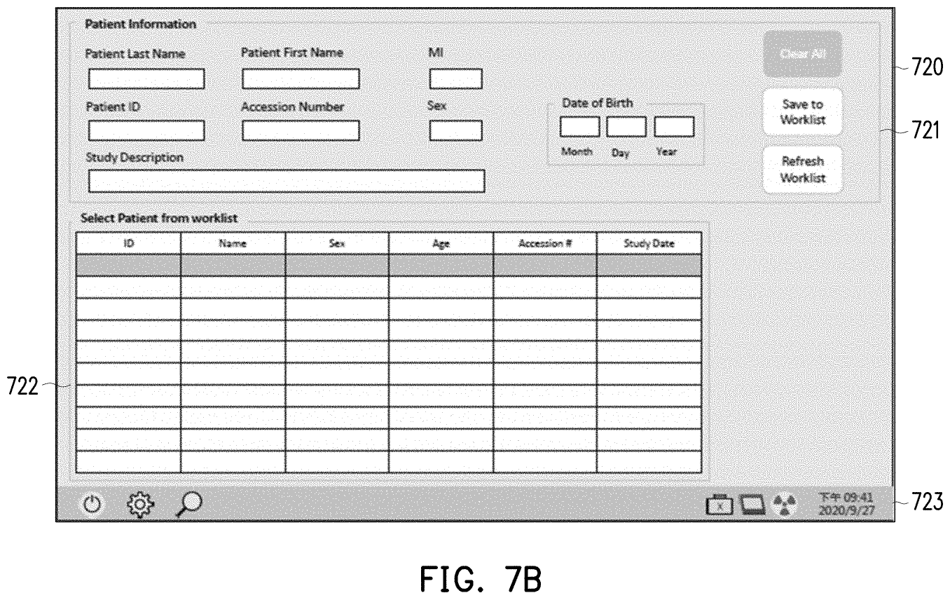

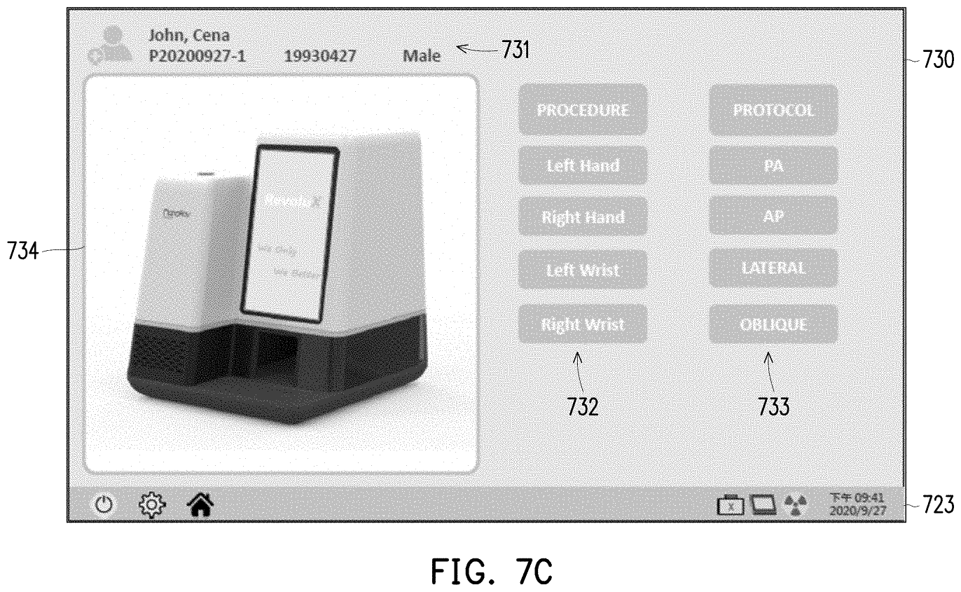

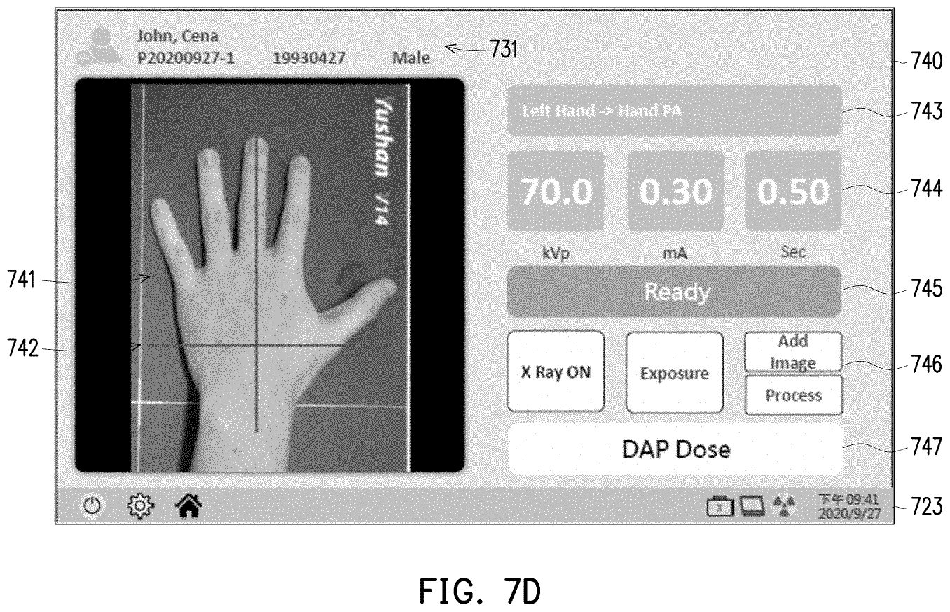

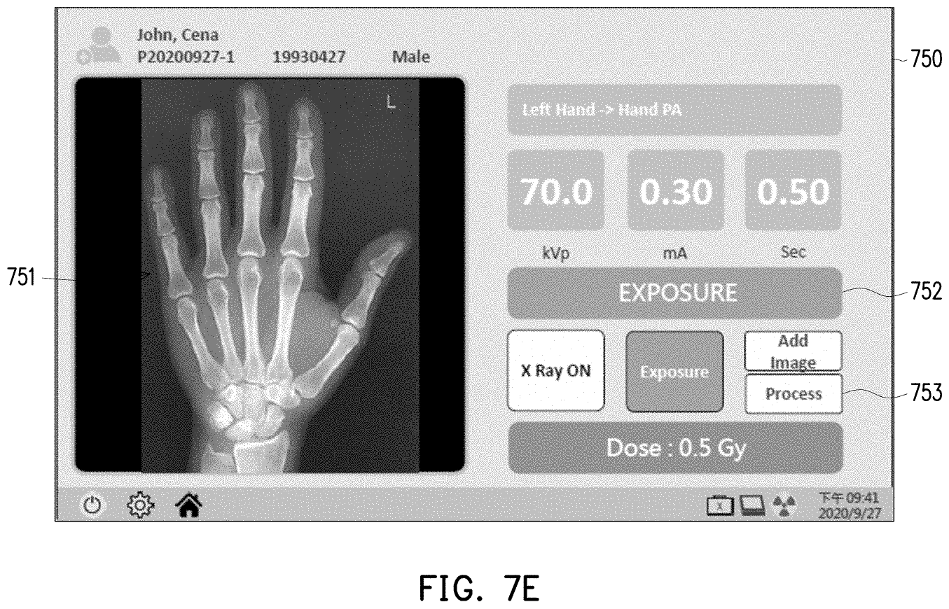

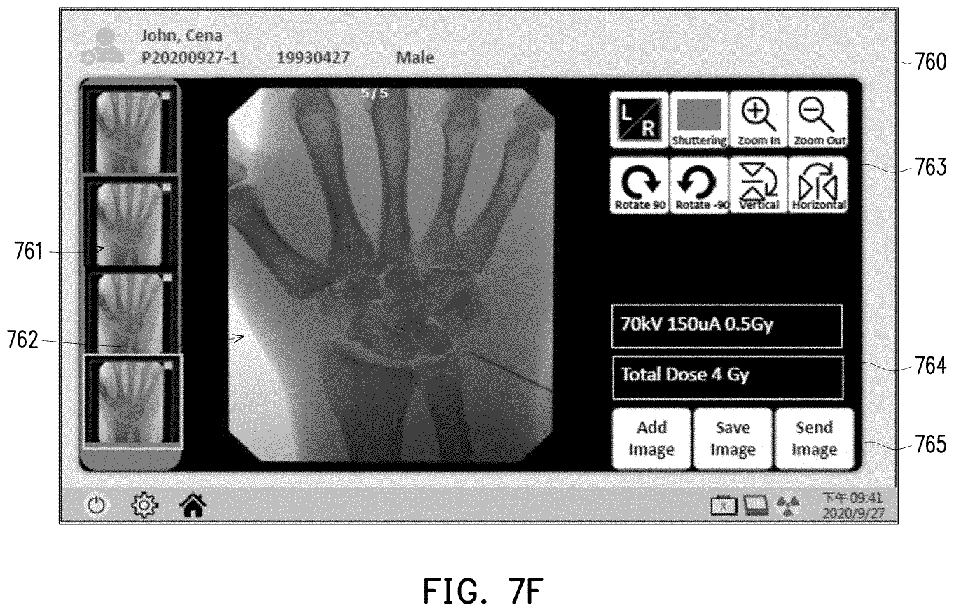

FIG. 6 is a flow chart of a radiation imaging method according to another embodiment of the disclosure. Referring to FIGS. 3 to 6, the radiation imaging system 300 of this embodiment may execute the following steps S601 to S609. In step S601, the operator may turn on the imaging device 310. The imaging device 310 is connected to the computer device 330. Referring to FIG. 7A, FIG. 7A is a schematic view of a login interface according to an embodiment of the disclosure. In step S602, the operation module 331 of the computer device 330 may display a login interface 710 on the display screen 332. As shown in FIG. 7A, the login interface 710 may include a verification area 711, a connection status bar 712, and a welcome screen (or a LOGO screen) 713. In this embodiment, the verification area 711 allows the operator to enter an account and a password to log in to confirm that the operator has sufficient access rights. In this embodiment, the connection status bar 712 may display the booting progress and the connection status of the imaging device 310. In this embodiment, the welcome screen 713 may include a model of the imaging device 310 or the manufacturer's trademark.

Referring to FIG. 7B, FIG. 7B is a schematic view of a diagnostic object information interface according to an embodiment of the disclosure. In step S603, when the operator performs a login operation through an input apparatus of the computer device 330 to allow the operation module 331 to complete the login operation, the operation module 331 of the computer device 330 may display a diagnostic object information interface 720 on the display screen 332. As shown in FIG. 7B, the diagnostic object information interface 720 may include a data input area 721, a subject list 722, and a status area 723. The data input area 721 may be used to input diagnostic object (subject) information. The diagnosis object information may include a variety of information such as name, date of birth, and notes. In this embodiment, the operator may input relevant data through the input apparatus of the computer device 330, so that the operation module 331 may obtain the diagnostic object information through the diagnostic object information interface 720. Alternatively, in an embodiment, the operator may scan a bar code on a relevant medical record or body data of the subject through the bar code scanner 333 of the operating computer device 330 to obtain the corresponding diagnostic object information.

In this embodiment, the subject list 722 may include a plurality of pieces of diagnostic object information to allow the operator to select and view specific diagnostic object information. The status area 723 may display the status of each element in the current imaging device 310 and the computer device 330. For example, the status area 723 may display the whole machine state information of the radiation source 312 of the imaging device 310, the mechanical shutter 318, the flat panel detector 313, and the display device 316. In addition, the status area 723 may further display the display screen 332 of the computer device 330 and a current network communication connection status.

Referring to FIGS. 7C and 7D, FIG. 7C is a schematic view of an operation interface according to an embodiment of the disclosure. FIG. 7D is a schematic view of another operation interface according to an embodiment of the disclosure. In step S604, after the operator completes the input of the diagnostic object information, the operating operation module 331 may display an operation interface 730 on the display screen 332 of the computer device 330. As shown in FIG. 7C, the operation interface 730 may include subject information 731, a shooting part area 732, a shooting positioning area 733, and an example screen 734. The subject information 731 may include the subject information of the current subject. The shooting part area 732 may include, for example, icons of parts available for shooting, such as left hand, right hand, left wrist, and right wrist. The shooting positioning area 733 may, for example, include various icons of positions such as PA, AP, lateral, and oblique. For example, the subject may enter the hand from the cavity opening 301 and place the hand in the radiation irradiation area 310D of the radiation isolation cavity 310C. Next, the subject may select appropriate icons of parts and icons of positions from the shooting part area 732 and the shooting positioning area 733 to record the current posture of the object under test to be shot. In addition, the example screen 734 may display an exemplary screen corresponding to the current part and position. In this embodiment, when the operator completes the selection of the shooting part and shooting position, the operation module 331 switches to display an operation interface 740 as shown in FIG. 7D on the display screen 332 of the computer device 330.

As shown in FIG. 7D, the operation interface 740 may include a video image 741, a registration pattern 742, part and positioning information 743, a radiation setting parameter 744, exposure operational status information 745, a control button area 746, and radiation dose information 747 of an object under test. The video image 741 may be a real-time image in the radiation isolation cavity 310C captured by the camera device 315. The registration pattern 742 may be a cross pattern projected by the infrared laser 317, but the disclosure is not limited thereto. For example, the operator may view the position and posture of the subject's hand in the radiation irradiation area 310D through the video image 741, and may determine whether the position and posture are correct based on the cross pattern projected on the subject's hand. In this embodiment, the part and positioning information 743 may present the part and position selected by the operator in the previous operation interface 730. The radiation setting parameter 744 may display and adjust the driving parameter of the radiation source 312. The exposure operational status information 745 may display the current operating status of the radiation source 312; for example, the exposure operational status information 745 may indicate that the radiation source 312 is currently in a status ready for exposure. The control button area 746 may be used to select the action to be performed currently. The action may include, for example, turning on the radiation source 312, performing exposure, increasing an exposure image, image processing, etc. The radiation dose information 747 may display the radiation dose detection result provided by the radiation dose detector 314.

In step S605, the operator may operate the control button area 746 of the operation interface 740 to turn on the radiation source 312. It is worth noting that the radiation source 312 enters the preparation for exposure (for example, 10 seconds) to be turned on and provide a continuous X-ray beam. In step S606, during the preparation for exposure, the operator may operate the hand-held switch 322 to execute exposure. In this regard, the mechanical shutter 318 may adjust the X-ray beam of the radiation source 312, and when the sensing pixel of the flat panel detector 313 is exposed, the mechanical shutter 318 may allow the X-ray beam of the radiation source 312 to be emitted to the object under test in the radiation irradiation area at the same time, and allow at least part of the beam to pass through the object under test, so that the flat panel detector 313 may obtain the radiation image of the object under test.

Next, the imaging device 310 may transmit the radiation image to the computer device 330. Referring to FIG. 7E, FIG. 7E is a schematic view of an operation interface according to yet another embodiment of the disclosure. In step S607, the computer device 330 may display a radiation image 751 in an operation interface 750. As shown in FIG. 7E, the operation interface 750 may include the radiation image 751, exposure operational status information 752, and a control button area 753. The radiation image 751 may display an image that has just been captured. The exposure operational status information 752 may display a current operating status of the radiation source 312; for example, the exposure operational status information 752 may indicate that the radiation source 312 is currently in the exposed status. The control button area 753 may be used to display the action that has just been completed or to select the action currently to be performed.

Referring to FIG. 7F, FIG. 7F is a schematic view of an image processing interface according to an embodiment of the disclosure. In step S608, the operator may operate the control button area 753 of the operation interface 750 to perform image processing, so that the operation module 331 may display an image processing interface 760 on the display screen 332. As shown in FIG. 7F, the image processing interface 760 may include an image list 761, a radiation image 762, an adjusting tool area 763, shot information area 764, and a control button area 765. The image list 761 may display a plurality of radiation images that have just been captured. It is worth noting that the radiation images refer to a plurality of images captured by the flat panel detector 313 during a plurality of periods of preparation for exposure. The flat panel detector 313 only captures one shot during one period of preparation for exposure to obtain one radiation image. In this embodiment, the radiation image 762 may be a specific radiation image selected from the image list 761 by the operator. The adjusting tool area 763 may include buttons for various image processing functions such as zooming in, zooming out, rotating, and cropping. The shot information area 764 may include shooting parameter information and radiation dose information corresponding to the selected specific radiation image. The control button area 765 may be used to select the action to be currently performed. The action may include increasing an image, saving an image, and transmitting an image, etc.

In this embodiment, the operator may operate the adjusting tool area 763 of the operation interface 750 to modify the radiation image 762. After the operator finishes modifying the radiation image 762, the operator may operate the control button area 765 of the operation interface 750 to store and transmit the image. In step S609, the operation module 331 may perform profiling and upload a detection file. In this embodiment, the operation module 331 may perform profiling based on the diagnostic object information and the radiation image 760 to generate the detection file. In addition, the computer device 330 may transmit the detection file to the cloud server 340, so that the artificial intelligence diagnostic module 341 of the cloud server 340 analyzes the detection file to automatically generate corresponding diagnostic data.

Therefore, through the radiation imaging system 300 and the radiation imaging method of the disclosure, a radiation image may be easily and quickly obtained, and after the computer device 330 performs real-time image processing on the radiation image, the radiation image may be uploaded to the artificial intelligence diagnostic module 341 of the cloud server 340 for image identification and analysis to automatically generate corresponding diagnostic data.

In summary, through the radiation imaging system and the radiation imaging method of the disclosure, an imaging device may be operated through a computer device so as to obtain a radiation image, and after image optimization processing is performed on the radiation image through the computer device, the radiation image after image optimization may be uploaded to the cloud system for automatic image analysis and diagnosis, so as to generate corresponding diagnostic data. The imaging device of the disclosure has the characteristics of small size and low radiation dose, so the imaging device may be applied to various detection environments and detection objects. In addition, the radiation imaging system of the disclosure has the advantages of being able to be set up quickly and having low requirements for the detection environment and apparatus.

Although the disclosure has been disclosed in the above by way of embodiments, the embodiments are not intended to limit the disclosure. Those with ordinary knowledge in the technical field can make various changes and modifications without departing from the spirit and scope of the disclosure. Therefore, the protection scope of the disclosure is subject to the scope of the appended claims.

What is claimed is:

1. A radiation imaging system, comprising:
a remote-control module; and
an imaging device, having a radiation isolation cavity, wherein the radiation isolation cavity comprises a radiation irradiation area adapted for placing an object under test, wherein the imaging device comprises:
a controller, coupled to the remote-control module;
a radiation source, coupled to the controller, disposed on a top of the radiation isolation cavity, wherein the radiation source faces the radiation irradiation area; and
a flat panel detector, coupled to the controller, disposed below the radiation irradiation area,
wherein during a preparation for exposure, the controller turns on the radiation source, and when the controller receives an activation signal output by the remote-control module, the controller operates the flat panel detector to obtain a radiation image corresponding to the object under test.

2. The radiation imaging system according to claim 1, wherein the imaging device further comprises:
a mechanical shutter, coupled to the controller,
wherein the radiation source is a continuous X-ray light source, and the controller operates the mechanical shutter to adjust an X-ray beam emitted by the radiation source.

3. The radiation imaging system according to claim 1, wherein the imaging device further comprises:
a radiation dose detector, coupled to the controller, disposed in the radiation isolation cavity, wherein the radiation dose detector is used to operate synchronously with the flat panel detector to obtain radiation dose information corresponding to the radiation image.

4. The radiation imaging system according to claim 1, wherein the remote-control module comprises:
a system switch, coupled to the controller, used to provide a switching signal to turn on the imaging device through the switching signal; and
a hand-held switch, coupled to the system switch, used to provide the activation signal.

5. The radiation imaging system according to claim 1, wherein the imaging device further comprises:
a camera device, coupled to the controller, disposed on the top of the radiation isolation cavity, wherein the camera device faces the radiation irradiation area and is used to obtain a video image corresponding to the object under test; and
a display device, coupled to the controller, used to display the video image.

6. The radiation imaging system according to claim 5, wherein the imaging device further comprises:
an infrared laser, coupled to the controller, disposed on the top of the radiation isolation cavity, wherein the infrared laser is used to emit a registration pattern toward the radiation irradiation area so that the video image comprises the registration pattern.

7. The radiation imaging system according to claim 5, further comprising:
a computer device, coupled to the imaging device, comprising a display screen,
wherein the computer device is used to execute an operation module, and an operation interface is displayed on the display screen,
wherein the computer device first displays the video image in the operation interface during the preparation for exposure, and after the flat panel detector obtains the radiation image, the computer device displays the radiation image in the operation interface,
wherein the operation interface further comprises radiation dose information, whole machine state information, and radiation setting parameter information.

8. The radiation imaging system according to claim 7, wherein when a processing icon in the operation interface is being executed, the operation module displays an image processing interface on the display screen, the image processing interface comprises the radiation image and a plurality of image processing function icons, and the operation module correspondingly modifies the radiation image according to an execution result of the image processing function icons.

9. The radiation imaging system according to claim 7, wherein the operation module displays a login interface on the display screen in advance, and when the operation module completes a login operation, the operation module displays a diagnostic object information interface on the display screen,
wherein the operation module obtains diagnostic object information through the diagnostic object information interface.

10. The radiation imaging system according to claim 9, wherein the operation module performs a profiling operation according to the diagnostic object information and the radiation image to generate a detection file, and the computer device communicates with a cloud server,
wherein the computer device transmits the detection data file to the cloud server, so that an artificial intelligence diagnostic module of the cloud server analyzes the detection file.

11. A radiation imaging method, adapted for a radiation imaging system, wherein the radiation imaging system comprises a remote-control module and an imaging device, the imaging device has a radiation isolation cavity, and the radiation isolation cavity comprises a radiation irradiation area adapted for placing an object under test, wherein the radiation imaging method comprises:
during a preparation for exposure, turning on a radiation source disposed on a top of the radiation isolation cavity by a controller;
during the preparation for exposure, outputting an activation signal by the remote-control module; and
when the controller receives the activation signal, operating a flat panel detector disposed below the radiation irradiation area by the controller to obtain a radiation image corresponding to the object under test.

12. The radiation imaging method according to claim 11, wherein the radiation source is a continuous X-ray light source, and the radiation imaging method further comprises:
operating a mechanical shutter by the controller to adjust an X-ray beam emitted by the radiation source.

13. The radiation imaging method according to claim 11, further comprising:
operating a radiation dose detector disposed in the radiation isolation cavity synchronously with the flat panel detector to obtain radiation dose information corresponding to the radiation image.

14. The radiation imaging method according to claim 11, further comprising:
providing a switching signal by a system switch of the remote-control module to turn on the imaging device through the switching signal; and
providing the activation signal by a hand-held switch of the remote-control module.

15. The radiation imaging method according to claim 11, further comprising:
obtaining a video image corresponding to the object under test by a camera device disposed on the top of the radiation isolation cavity; and
displaying the video image by a display device.

16. The radiation imaging method according to claim 15, further comprising:
emitting a registration pattern toward the radiation irradiation area by an infrared laser disposed on the top of the radiation isolation cavity, so that the video image comprises the registration pattern.

17. The radiation imaging method according to claim 15, wherein the radiation imaging system further comprises a computer device, and the radiation imaging method further comprises:
executing an operation module by a computer device, and displaying an operation interface on a display screen of the computer device;
during the preparation for exposure, displaying the video image in the operation interface by the computer device first; and
after the flat panel detector obtains the radiation image, displaying the radiation image in the operation interface by the computer device;
wherein the operation interface further comprises radiation dose information, whole machine state information, and radiation setting parameter information.

18. The radiation imaging method according to claim 17, further comprising:
when a processing icon in the operation interface is being executed, displaying an image processing interface on the display screen by the operation module, wherein the image processing interface comprises the radiation image and a plurality of image processing function icons; and
correspondingly modifying the radiation image by the operation module according to an execution result of the image processing function icons.

19. The radiation imaging method according to claim 17, further comprising:
displaying a login interface on the display screen in advance by the operation module;
after the operation module completes a login operation, displaying a diagnostic object information interface on the display screen by the operation module; and
obtaining diagnostic object information through the diagnostic object information interface by the operation module.

20. The radiation imaging method according to claim 19, further comprising:
performing a profiling operation by the operation module according to the diagnostic object information and the radiation image to generate a detection file;
communicating with a cloud server by the computer device;
transmitting the detection data file to the cloud server by the computer device; and
analyzing the detection file by an artificial intelligence diagnostic module of the cloud server.
